(19) United States
(12) Patent Application Publication
(10) Pub. No.: US 2002/0168371 A1
(43) Pub. Date: Nov. 14, 2002

(54) Process for reducing antibody response against xenografts

(76) Inventor: Michel Awwad, Hudson, NH (US)

Correspondence Address: Alan J. Grant, Esq., c/o Carella, Byrne, Bain, Gilfillan,, Cecchi, Stewart & Olstein, 6 Becker Farm Road, Roseland, NJ, 07068, US

(21) Appl. No.: 09/861,056

(22) Filed: May 18, 2001

Related U.S. Application Data
(60) Provisional application No. 60/205,046, filed on May 18, 2000.

(51) Int. Cl. A61K 39/395; A61K 38/38; A61K 31/715

(52) U.S. Cl. 424/178.1; 514/8; 514/54

(57) ABSTRACT

A process for reducing in a primate recipient a natural antibody response against a graft from a non-primate donor, comprising administering to said recipient an effective amount of a carrier linked to one or more galactosyl-α-1,3-galactose moieties is disclosed, along with additional optional processes involving use of immunosuppressive agents and hematopoietic stem cells.

This application claims priority of U.S. Provisional Application 60/205,046, filed May 18, 2000, the disclosure of which is hereby incorporated by reference in its entirety.

### FIELD OF THE INVENTION

This invention relates to the depletion or down-modulation of natural antibodies responsible for xenogeneic antibody-mediated graft rejection.

### BACKGROUND OF THE INVENTION

Organ transplantation has become a well established clinical procedure. However, there are still several major problems that must be resolved in order to provide a satisfactory outcome to all potential transplant recipients. These include the lack of available donor organs and the need for long-term immunosuppression to prevent graft rejection.

The need for organs has continued to rise. Currently, there are more than 69,000 Americans waiting for organ transplant, but only about 22,000 organ transplants are performed per year. Because of this widening gap, xenogeneic organ transplantation is an increasingly important area of interest.

Size, physiological similarities, availability and ethical issues have made the pig one of the best studied organ donor species for xenotransplantation (Sachs, D. H., “MHC-Homozygous Miniature Swine” in Swine as Models in Biomedical Research, Swindle, M. M. et al (Eds), Iowa State Univ. Press, Ames, Iowa (1992); Cooper, D. K. C. et al, “The Pig as Potential Organ Donor for Man in Xenotransplantation” Cooper, D. K. C. et al (Eds.), Springer-Verlag, Heidleberg, Germany (1991)).

Graft rejection may be a consequence of either, or both, cell-mediated and antibody-mediated events. Based upon results of histopathology tests, graft rejection has been characterized to be hyperacute, acute or chronic. Antibody-mediated rejection may be involved in all of these stages or rejection (Chapter 13 of “Cellular and Molecular Immunology,” 3^{rd} Edition, Abbas, A. K. et al (Eds.), Saunders & Co., Philadelphia, Pa.).

Hyperacute rejection is characterized by rapid thrombotic occlusion of the graft vasculature that begins within minutes to hours after host blood vessels are anastomosed to graft vessels. Hyperacute rejection is mediated by antibodies that pre-exist in naïve hosts, the so-called “natural antibodies,” which bind to endothelium and activate complement. Antibody and complement induce a number of changes in the graft endothelium that promote intravascular thrombosis.

Acute vascular or delayed graft rejection, like hyperacute rejection, is characterized by interstitial edema and hemorrhage. However, in acute vascular rejection the extent of thrombosis is more pronounced, and there is an infiltrate consisting of mononuclear leukocytes and neutrophils. Acute vascular rejection is observed in both allografts and xenografts. While the mechanism underlying acute vascular rejection is not well understood, natural antibodies are considered to play a significant role. Elimination of natural antibodies would therefore facilitate the engraftment of transplanted hematopoietic stem cells (HSC) thereby enabling the induction of a state of mixed hematopoietic chimerism, which in turn should lead to the induction of specific immune tolerance to the donor organ transplant, whether it be an allograft or a xenograft. This would serve to avoid the use of immunosuppressive agents and other long-term debilitating treatments and, in the case of xenotransplantation, overcome the deficiency in the number of donor organs available for organ transplants.

Natural antibodies are believed to arise as a consequence of exposure to cross-reactive microbial antigens. In the allogeneic setting, the natural antibodies are directed toward the red blood cell surface antigens, described as ABO antigens. Differences in the ABO system between donors and recipients limit blood transfusions and organ transplants by causing antibody and complement-mediated dependent cell lysis of cells expressing the blood group antigens. Blood transfusion techniques avoid these problems by matching blood types. However, hyperacute rejection of allografts may still occur as a consequence of natural antibodies specific for other alloantigens.

Xenogeneic natural antibody-mediated hyperacute rejection is a very significant barrier to xenotransplantation (Platt et al, Transplantation, 52: 937 (1991)) and overcoming this barrier is important to the long-term success of pig-to-primate xenotransplantation. A predominant epitope on porcine cells recognized by human natural antibodies is a carbohydrate that includes a terminal galactose residue in the conformation of the galactosyl-α-1,3-galactose disaccharide structure (Neethling et al, Transplantation, 57:959 (1994); Ye et al, Transplantation, 58:330 (1994); Sandrin et al, Proc. Natl. Acad. Sci. USA, 90:11391 (1993); Good et al, Trans. Proc., 24:559 (1992)) Immunopathologic analysis of tissue samples from organs undergoing hyperacute rejection reveals the presence of recipient natural antibodies and complement components along the epithelial surfaces of blood vessels. (Leventhal et al, Transplantation, 2:14 (1993); Platt et al, Transplantation, 56:14 (1991); Platt et al, Transplantation, 52:14 (1991); Platt et al, Transplantation, 52:37 (1991); Galili et al, J. Exp. Med., 160:1519 (1984)).

Sandrin et al (1993) found that it is the IgM class of natural antibodies, most of which are specific for the Gal-α-1,3-Gal determinant (herein, the Gal determinant or just Gal) that initiate the activation of human complement on porcine cells. The Gal epitope is synthesized by addition of a terminal galactosyl residue to a pre-existing galactose residue linked to an N-acetyl-glucosaminyl residue in a reaction catalyzed by glucosyltransferase-UDP-galactose: α-D-galactosyl-1,4-Nacetyl-D-glucosamide-α-1,3-galactosyltransferase (α-1,3-GT). In species expressing α-1,3-GT, natural antibodies reactive against the Gal-epitopes are absent. The lack of α-1,3-GT in humans, apes, and Old World primates results in a failure to express the Gal epitope, making the expression of antibodies reactive with this epitope permissible. In mice, a species that normally expresses the Gal epitope, disruption of the murine α-1,3-GT gene using embryonic stem cell technology leads to the development of natural antibodies reactive against the Gal-α-1,3-Gal (Gal) moiety. (See: Thall et al, J. Biol. Chem., 270:21437 (1995); Thall et al, Trans. Proc., 28:561 (1996))

Several approaches have heretofore been explored in pig-to-primate experimental models in an effort to overcome hyperacute rejection. In one approach, recipient natural antibodies are depleted by organ perfusion or by immunoadsorption over Gal immunoaffinity columns and hyperacute rejection of the transplanted organ is delayed or does not occur. However, the natural antibodies return to the pre-depletion levels within about a week and organ rejection resumes. In other approaches, the depletion or inhibition of complement or the use of organs from genetically engineered pigs transgenic for human complement regulatory proteins have also been successfully used to overcome hyperacute rejection. However, in almost every case, all of these manipulations have been combined with heavy pharmacologic immunosuppressive therapy which, while ineffective by itself, extends graft survival if hyperacute rejection is prevented. Despite this, in those graft recipients that have survived the therapeutic regimen, graft failure has resulted almost invariably from what is believed to be an antibody-mediated form of rejection rather than cellular rejection. (See: Latinne et al, Trans. Proc., 250:336 (1993))

Protocols in xenotransplantation have heretofore included the step of removing the anti-Gal antibodies prior to transplantation of the HSCs. Because of the rapid return of the natural antibodies it is desirable to keep the level of these to a minimum. Since very little is known about the cells that make the anti-Gal antibodies, effective strategies to inhibit production of these antibodies have not yet proven successful for a sufficient length of time to enable administered HSCs to engraft.

In sum, antibodies (Abs) in primates directed against Gal-α-1-3-Gal (Gal) determinants on pig cells form a major barrier to the successful xenotransplantation of cells or organs in the pig-to-primate model (1-5) although these can be selectively depleted by extra-corporeal immunoadsorption (EIA) of plasma through immunoaffinity columns of a Gal oligosaccharide (6-9). Using a course of 3 EIAs in baboons, anti-Gal IgM and IgG can be depleted by >97% and >99%, respectively (10). While this prevented hyperacute rejection, return of antibodies resulted in humoral rejection (acute vascular/delayed xenograft rejection or AVR/DXR) occurring within a few days (9,11,12). Intensive pharmacological therapy and/or complement depletion or inhibition can delay the onset of this humoral-mediated rejection further, but is unable to prevent it.

Induced antibodies to both Gal and non-Gal epitopes may play a role in the development of AVR/DXR. When the induced Ab response is prevented by therapy with an anti-CD154 monoclonal antibody (mAb) (13,14), however, a transplanted pig organ is still at risk from the return of natural Ab, particularly Gal-reactive IgM, which may be associated with the development of AVR/DXR and disseminated intravascular coagulation, necessitating urgent graft excision (11,12,15-18). In the pig-to-primate model, no therapy has to date either prevented return of natural Ab or completely suppressed its production (19).

Continuous intravenous (i.v.) infusion of a source of antigen or oligosaccharide bound by Gal-reactive antibody (Ab) to prevent binding of Ab to a transplanted pig organ or cells has proven successful in regard to the prevention of Ab-mediated rejection of ABO-incompatible renal allografts in baboons (22). The continuous i.v. infusion of synthetic A or B trisaccharides, which mimic the terminal sugar molecules of the respective epitopes, enabled rejection to be prevented in this model. Similar studies in the pig-to-baboon model, using natural or synthetic Gal saccharides, proved less successful (6,23,24).

The present invention solves these problems through the process of infusing Gal saccharides conjugated to bovine serum albumin. Bovine serum albumin conjugated to Gal type 6 (BSA-Gal) has been used as the target in an ELISA to measure serum levels of Gal-reactive IgM and IgG (8,9,25). The present invention uses BSA-Gal to adsorb anti-Gal Ab, whereby the BSA-Gal-Ab complex is probably cleared from the circulation largely in the liver (26-29), but also possibly in the lungs and kidneys (26,29). Earlier investigation of BSA-Gal for this purpose was delayed by two concerns, namely, (i) that the binding of Ab to BSA-Gal would result in the formation of immune complexes that would cause injury to the kidneys or liver, and (ii) that sensitization to BSA-Gal would develop, negating any beneficial effect within a few days. However, the ability of anti-CD154 monoclonal antibody (MAb) therapy to prevent sensitization to Gal and non-Gal antigens facilitates prevention of any induced Ab response to BSA-Gal. (In humans, the use of human serum albumin (HSA)-Gal conjugate would not cause sensitization. In the baboon, even HSA results in sensitization if administered to a non-immunosuppressed animal.

### BRIEF SUMMARY OF THE INVENTION

In one aspect, the present invention relates to a process for reducing the adverse effects of anti-gal antibodies in xenotransplantation wherein the recipient of a xenotransplant is treated with a plurality of galactosy-α-1,3-galactose moieties (herein referred to as “Gal”) linked, chemically or otherwise, to a pharmaceutically acceptable carrier. In specific embodiments, the carrier or support is a polymer, such as a synthetic polymer or naturally occurring polymer such as a protein, preferably wherein said carrier is bovine serum albumin (BSA) or human serum albumin (HSA).

In a preferred embodiment, the present invention relates to a process of using the carrier-linked-Gal moieties of the present invention as part of a regimen for promoting the acceptance of a transplant, especially a xenotransplant, including cells, tissues or whole organs, wherein there is also administered to the recipient as part of the same regimen hematopoietic stem cells (for example, a bone marrow preparation derived from the donor). In one such preferred embodiment, the donor is a swine and the recipient is a primate, especially a human being.

In another embodiment, the recipient is also treated with an immunosuppressive agent (to prevent sensitization to the Gal-carrier moiety). In preferred embodiments, this agent may be an inhibitor of a co-stimulator pathway, or a T cell depletory (for example, an anti-CD2 antibody, such as MEDI-507) or a combination of both.

In one embodiment of the present invention, there is provided a process for promoting acceptance by a recipient mammal of a graft from a donor mammal of a different species (i.e., a xenogeneic graft) comprising administering to the recipient an immunoadsorbent of anti-Gal antibodies, such as a Gal-linked polymer, an immunosuppressive agent and hematopoietic stem cells, which can then be followed by implanting of the graft into the recipient.

### BRIEF DESCRIPTION OF THE DRAWINGS

FIG. 1. Terminal molecular structures of Gal type 2 and type 6 saccharides.

FIG. 2. Basic nonmyeloablative regimen for baboons in Groups 1 and 2.

SPX=splenectomy; WBI=whole body irradiation in two fractions (each of 150 cGy); TI=thymic irradiation (700 cGy); ATG=anti-thymocyte globulin; EIA=extracorporeal immunoadsorption; MMF=mycophenolate mofetil (by continuous i.v. infusion); CyA=cyclosporine (by continuous i.v. infusion); PHM=prostacyclin, heparin, methylprednisolone; pIL3+pSCF=porcine hematopoietic growth factors. Transplantation of pig PBPC was on days 0, 1 and 2. In 2 of the Group 1 baboons, 2 doses of anti-CD154 mAb were added to the basic regimen on days 0 and 2. In the other 2 Group 1 baboons, CyA was replaced by a course of anti-CD154 mAb, given on alternate days from day 0 to day 14 (8 doses). Group 2 baboons received the same therapy as those in Group 1 (including CyA) but received anti-CD154 mAb on alternate days for 29-43 days. In addition, Group 2 received a continuous i.v. infusion of BSA-Gal from days 0 to 19-30 (not shown).

FIG. 3. Anti-HSA antibody levels in baboons before and after the i.v. administration of HSA with and without therapy with anti-CD154 mAb.

FIG. 4. (A) Anti-Gal (type 6) Ab levels in a baboon that underwent EIA on days −3, −2 and −1, followed by the continuous i.v. infusion of BSA-Gal at rates varying from 5-20 mg/kg/day (indicated) for 8 days. Anti-Gal IgM and IgG were rarely measurable in the serum during the administration of BSA-Gal. (B) Following the i.v. administration of a bolus of BSA-Gal (*500 mg over 2 hours) to a baboon, no anti-Gal (type 6) IgM or IgG could be measured in the serum. This state of absence of measurable anti-Gal antibody was maintained during the continuous i.v. infusion of BSA-Gal at 50 mg/kg/day (indicated) for 5 days (days 0-4). Once the infusion was discontinued, anti-Gal Ab began to be detected again in the serum (day 5).

FIG. 5. Levels of anti-Gal IgM and IgG reactive with both Gal type 6 and type 2 in representative baboons of Groups 1 (A) and 2 (B, C and D). The limit of detection by this ELISA for IgM is 5 μg/ml and for IgG is 0.5 μg/ml. The first day of porcine PBPC transplantation was on day 0, administered after the final EIA of the preparative regimen. In the Group 2 baboons (B-D), the dosages of BSA-Gal are indicated by the shaded bars. (A) In a Group 1 baboon (B57-323) that received anti-CD154 mAb×8 doses and no CyA, there was no increase in anti-Gal type 6 IgM or IgG over pre-PBPC levels with a very slightly “rebound” of anti-Gal type 2 IgM and IgG to just above pre-PBPC levels, indicating that no sensitization occurred. (B) In a Group 2 baboon (B69-331), the return of both type 6 and type 2 Gal-reactive Ab was delayed for 30 days until BSA-Gal therapy was discontinued, after which Ab to type 6 and type 2 Gal slowly increased, but remained at less than the pre-EIA levels. No sensitization occurred. (C) In one Group 2 baboon (B69-129), Ab reactive with Gal type 2 trisaccharide returned during BSA-Gal therapy even while Ab to Gal type 6 remained depleted. (In the two other Group 2 baboons, return of anti-Gal type 2 and type 6 Ab was synchronous.) (D) One Group 2 baboon (B69-256) developed induced Gal-reactive IgG (to both Gal type 6 and type 2) after cessation of anti-CD154 mAb therapy, believed to be due to the continuing presence of high levels of pig cell microchimerism. No Ab to non-Gal porcine determinants was detected.

FIG. 6. Changes in optical density of anti-Gal IgM and IgG reactive with type 6 and type 2 saccharides in the 3 baboons of Group 2. (A) IgM and (B) IgG reactive with Gal type 6. (C) IgM and (D) IgG reactive with Gal type 2. In B69-331, the level of IgM reactive with Gal type 6 rose on day 16 but, following an increase in the infusion rate of BSA-Gal (not shown; see FIG. 5B), fell to undetectable levels. In B69-129, Ab reactive with Gal type 2 (IgM and IgG) rose to approximate pre-EIA levels by day 15 despite BSA-Gal therapy that was sufficient to maintain depletion of Ab reactive with Gal type 6. After discontinuation of BSA-Gal therapy in B69-256, there was an increase in IgG reactive with both Gal types 6 and 2 to above pre-EIA levels, indicating some degree of sensitization.

FIG. 7. Changes in the numbers of CD3 (A) and CD20 (B) cells in Group 2 baboons. The horizontal shaded bar indicates maximum length of therapy with BSA-Gal. Following induction therapy with WBI and antithymocyte globulin, both CD3 and CD20 cells were largely depleted. Recovery of CD3 cells had occurred by the conclusion of the course of BSA-Gal therapy (days 19-30). Recovery of CD20 cells, which had been more efficiently depleted, was slow in 2 cases, particularly while BSA-Gal was being administered.

FIG. 8 shows generally how BSA-Gal can inhibit the binding of human serum (natural antibodies) to plates coated with BSA-Gal approximately 100 to 10,000 fold better than free sugar as determined by ELISA.

FIG. 9 shows the Anti-Gal profile of a baboon treated with BSA-Gal.

### DESCRIPTION OF THE INVENTION

In one aspect, the present invention relates to a process for reducing the adverse effects of anti-gal antibodies in transplantation procedures, especially xenotransplantation procedures, wherein the recipient of a xenotransplant is treated with a plurality of galactosy-α-1,3-galactose moieties (herein referred to as “Gal”) linked, chemically or otherwise, a pharmaceutically acceptable carrier. In specific embodiments, the carrier or support is a polymer, such as a synthetic polymer or naturally occurring polymer such as a protein, preferably wherein said carrier is bovine serum albumin (BSA) or human serum albumin (HSA). In particular, the process of the present invention is a process for reducing or eliminating the presence of anti-Gal antibodies for a time sufficient to promote acceptance of a transplant, especially a xenotransplant.

In one embodiment of the present invention, there is provided a process for promoting acceptance by a recipient mammal, especially a human being, of a graft from a donor mammal of a different species (i.e., a xenogeneic graft) comprising administering to the recipient an immunoadsorbent of anti-Gal antibodies, such as a Gal-linked polymer as disclosed herein, and optionally an immunosuppressive agent and/or hematopoietic stem cells, which can then be followed by implanting of the graft into the recipient.

In accordance with the present invention, neoglycoproteins containing the gal-α-1,3-gal (Gal) moiety may be selected from the group consisting of gal-α-1,3-gal linked to bovine serum albumin (BSA) and human serum albumin (HSA) using spacers of various lengths, e.g., structures available from Dextra Laboratories (U.K.), especially catalog numbers NGPO203 (an α1,3Gal-BSA Gal, 3 atom spacer), NGP1203 (Gal-α1,3Gal-BSA, 14 atom spacer) NGP2203 (Galα1,3Gal-HSA, 3 atom spacer) and NGP3203 (Galα1,3Gal-HSA, 14 atom spacer)

Glycoconjugates containing the gal-α-1,3-gal moiety may also be selected from the group consisting of gal-α-1,3-gal-α-1,4-glc or gal-α-1,3-gal-α-1,4-GIcNAc linked to BSA or HSA with spacers of different lengths (such as those mentioned above). A preferred such structure is Gal-α-1,3-Gal-β-1,4-Glc-X-Y (a linear B type 6 sugar) linked to BSA (available from Alberta Research Counsel, Canada).

In a preferred embodiment, the present invention relates to a process of using the carrier-linked-Gal moieties of the present invention as part of a regimen for promoting the acceptance of a transplant, especially a xenotransplant, including cells, tissues or whole organs, wherein there is also administered to the recipient an immunosuppressive agent (to prevent sensitization to the Gal-carrier moiety). In preferred embodiments, this agent may be an inhibitor of a co-stimulator pathway, or a T cell depletory (for example, an anti-CD2 antibody, such as MEDI-507, available from BioTransplant, Inc., Charlestown, Mass.) or a combination of both co-stimulatory blockade pathway and a T cell depletor or depleting agent. In preferred embodiments, such a co-stimulatory pathway inhibitor may be one or both of an inhibitor for CD40-CD154 interaction and a blocker of the CD28-B7 interaction. Such inhibitors may act to reduce or eliminate an immune response to the solid carrier for Gal and thus other agents also exhibiting this function could be equivalently used in the processes of the invention.

In a preferred embodiment, the CD40-CD154 interaction is inhibited by administering an antibody or soluble ligand or receptor for the CD154 or CD40 antigens, for example, by administering an anti-CD154 antibody, most preferably 5c8 (such as described in U.S. Pat. No. 5,474,711, the disclosure of which is hereby incorporated by reference in its entirety). In preferred embodiments, such an inhibitor binds to CD154.

In other preferred embodiments, the CD28-B7 interaction is inhibited by administering an antibody or soluble ligands or receptors for CD28 and/or B7, for example, a soluble CTLA4Ig, a CTLA4 fusion protein or immunoglobulin fusion protein. Preferably, the inhibitor binds B7. In preferred embodiments, anti-B7-1 and or anti-B7-2 antibodies are administered. (see, for example, Lenschow D J, Zeng Y, Thistlethwaite J R, Montag A, Brady W, Gibson M G, Linsley P S, Bluestone J A. Long-term survival of xenogeneic pancreatic islet grafts induced by CTLA4Ig. Science. (Aug. 7, 1992) 257(5071 ):789-92).

Thus, in a preferred embodiment, the present invention relates to a process for promoting acceptance and/or reducing rejection of a graft in a recipient mammal, especially a primate, most especially a human being, from a donor mammal, especially a pig, or other xenogeneic source, comprising administering to the recipient an immunoadsorbent of anti-Gal antibodies, especially galactosy-α-1,3-galactose (Gal), linked to a polymer, such as a protein, to form a polymer linked Gal epitope, administering to said recipient an immunosuppressive agent, especially one that prevents sensitization of the recipient to the Gal-callier, and administering to said recipient an effective amount of hematopoietic stem cells.

In specific preferred embodiments, said hematopoietic stem cells are derived from the donor (i.e., autologous) or from an animal of the same species as the donor (i.e., allogeneic). The source of such hematopoietic stem cells may be a bone marrow preparation or other source and said cells may be administered to said recipient by intravenous injection. The latter may, for example, serve to better prepare the recipient for acceptance of the subsequent graft by inducing tolerance at both the T cell and B cell levels.

In carrying out the processes of the invention, the carrier-Gal moieties, the immunosuppressive agent and the hematopoietic stem cells may be administered as separate compositions or as a single composition with timing not an essential feature except that these are administered prior to or contemporaneously or simultaneously with the graft or transplant. Administration of said agents following the graft or transplant is also specifically contemplated by the present invention.

In administering the above described components, each is to be understood as being administered in an effective amount for the purposes disclosed herein. Thus, the hematopoietic stem cells will be administered in an amount sufficient to reduce T cell and/or B cell responses to the transplant. The immunosuppressive agents will be administered in an amount sufficient to prevent, or at least greatly reduce, reaction to the carrier-Gal moiety and the Carrier-Gal moiety is administered in an amount sufficient to reduce, or completely prevent, graft or transplant rejection.

In accordance with the foregoing, the recipient of said treatments may be a mammal, such as a primate, including a human being. The donor may be another mammal, preferably a swine, most preferably a miniature swine. The graft is commonly from an otherwise non-compatible or discordant species, so that the transplant is xenogeneic. In a highly preferred embodiment, the recipient is a primate, especially a human, and the donor is a swine, especially a miniature swine.

An advantage of the present invention is the lack of requirement for administration of any hematopoietic space creating irradiation, such as whole body irradiation. In some embodiments, the process of the invention can be practiced with or without T cell depletion or inactivation, e.g., without the administration of thymic irradiation, or anti-T cell antibodies. The processes of the invention may also be practiced with partial T cell depletion or inactivation, e.g., by the administration of thymic irradiation, or anti-T cell antibodies and in such amounts as to result in partial depletion of recipient T cells.

In preferred embodiments, the processes of the invention include administering a sufficiently large number of donor hematopoietic stem cells to the recipient such that donor stem cells engraft and give rise to a mixed hematopoietic stem cell chimerism, without the need for hematopoietic space-creating irradiation. The donor cells can be provided in one, two or more administrations, either prior to or contemporaneously with (in some case, perhaps even after), the anti-Gal adsorbing agents (i.e., carrier-Gal). In preferred embodiments, mixed chimerism is induced in the recipient and the state of mixed chimerism is formed in the absence of the induction of hematopoietic space, e.g., in the absence of hematopoietic space created by space-creating irradiation (such as whole body irradiation).

In other preferred embodiments, natural killer cells are inactivated, preferably by graft reactive or xenoreactive, e.g., swine reactive NK cells of the recipient, such as an anti-CD2 antibody (such as that described in U.S. Pat. No. 5,730,979 or U.S. Pat. No. 5,951,983). The administration of such antibodies, or other treatment to inactivate natural killer cells, can be given prior to administering the hematopoietic stem cells to the recipient or subsequent to administering such cells but prior to transplanting the graft into the recipient. Such antibodies may be the same or different from antibodies used to inactivate T cells.

In preferred embodiments, ex vivo immunoadsorption of natural antibodies against the Gal epitope may be performed using processes like those described in U.S. Pat. No. 5,651,968 (hereby incorporated by reference).

In another embodiment, the present invention relates to a process for reducing rejection of a xenotransplant comprising administering to the recipient an effective amount of an immunoadsorbent of anti-Gal antibodies, preferably a Gal-α-1,3-Gal moiety linked to a carrier, such as a protein or other polymer, including synthetic polymers and supports, especially Gal-linked BSA, or Gal-linked HSA, and an inhibitor of a co-stimulatory response wherein, prior to or simultaneously with the transplantation or grafting, administering to said recipient a sample of donor thymic tissue, such as thymic epithelium, preferably fetal tissue, most preferably porcine fetal tissue, and, optionally, implanting the graft in the recipient, whereby the thymic tissue induces immunological tolerance at the T cell level.

In a preferred embodiment, ex vivo immunoadsorption of natural antibodies against the Gal epitope may be performed by techniques known in the art, such as that described in U.S. Pat. No. 5,651,968 (incorporated by reference in its entirety).

In another embodiment, the present invention relates to a process for reducing rejection of a xenotransplant comprising administering to the recipient an effective amount of an immunoadsorbent of anti-Gal antibodies, preferably a Gal-α-1,3-Gal moiety linked to a carrier, such as a protein or other polymer, including synthetic polymers and supports, especially Gal-linked BSA, or Gal-linked HSA, and an inhibitor of a co-stimulatory response wherein, prior to or simultaneously with the transplantation or grafting, said recipient receives a transplant of a composite thymo-organ, such as a thymokidney or thymoheart.

As disclosed herein, a composite thymo-organ is created by implanting thymic autografts into the donor organ and allowing the implanted donor thymic tissue to be transplanted as part of a vascularized organ. This prior vascularization of the thymic tissue allows the thymic tissue to become functional immediately after transplant thereby facilitating the development of tolerance to the donor antigens.

In another aspect, the present invention relates to a process for reducing rejection and/or promoting acceptance by a recipient mammal, such as a primate, especially a human being, comprising administering to the recipient an immunoadsorbent of anti-Gal antibodies (for example, a multimeric form of the Gal-α-1.3-Gal moiety, such as Gal-BSA, and an inhibitor of a co-stimulatory pathway or interaction, and transplanting of a composite thymo-organ (such as thymokidney or thymoheart) further comprising islet cells (an islet-thymo-organ).

As used herein, an islet-thymo-organ is created by implanting thymic and islet autografts into the donor organ and allowing the implanted donor thymic tissue and islets to be transplanted as part of a vascularized organ. Such prior vascularization of the thymic tissue allows the thymic tissue to become functional immediately after transplant thereby facilitating the development of tolerance to the donor antigens and provides functional islet cells at the same time.

### General Materials and Methods

### Animals

Baboons (Papio anubis, n=9) of known ABO blood group and of body weight 8-14 kg (Biological Resources, Houston, Tex.) were used as recipients. Massachusetts General Hospital MHC-inbred miniature swine (n=6) of blood group O, 2-6 months old, 18-60 kg of body weight (Charles River Laboratories, Wilmington, Mass.) served as donors of mobilized peripheral blood leukocytes that contained approximately 2% progenitor cells (PBPC) (30). All experiments were conducted according to the NIH Guidelines for Care and Use of Laboratory Animals and were approved by the Massachusetts General Hospital Subcommittee on Research Animal Care.

### Surgical Procedures and Mobilization and Collection of Porcine Progenitor Cells (PBPC)

Anesthesia, i.v. line placement in pigs and baboons, and intra-arterial line placement and splenectomy in baboons utilize known protocols (12,31), as has hematopoietic growth factor-mobilization of porcine progenitor cells and leukapheresis (30). Leukapheresis resulted in the collection of large numbers of leukocytes (30-60×10¹⁰ cells) containing approximately 2% progenitor cells (designated mobilized peripheral blood progenitor cells, PBPC) (30).

### Extracorporeal Immunoadsorption in Baboons

Anti-Gal Ab was depleted from the baboon's circulation by the perfusion of plasma through immunoaffinity columns containing synthetic Galα1-3Galβ1-4GIc-X-Y (αGal type 6 trisaccharide, ARC), as reported previously (8-10,25).

### Conditioning Regimen in Baboons

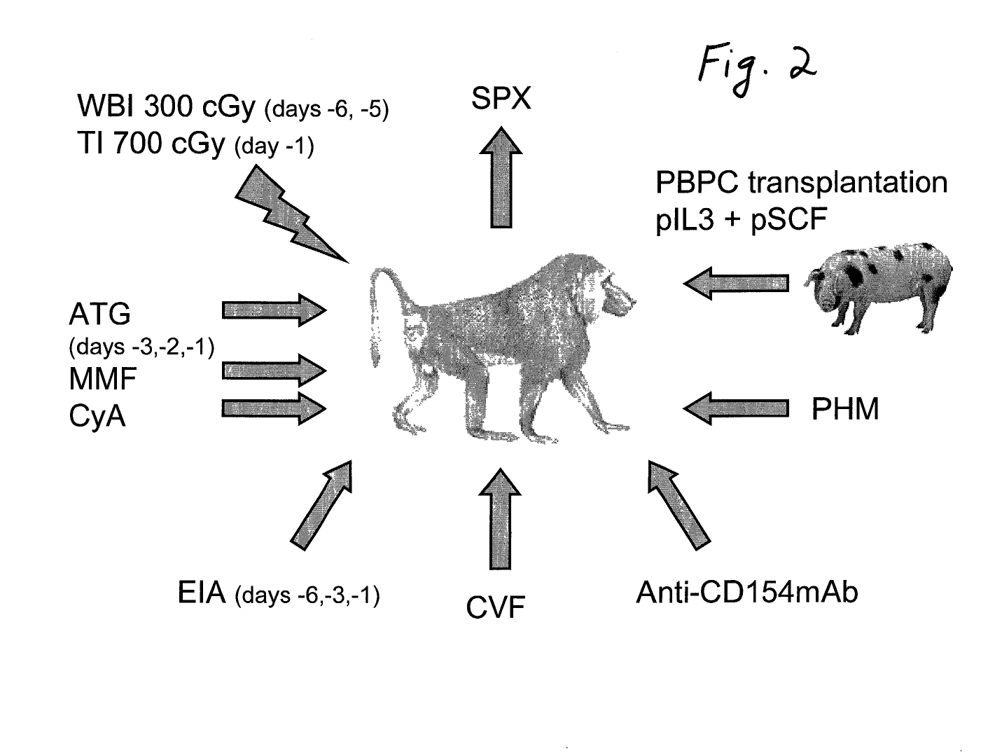

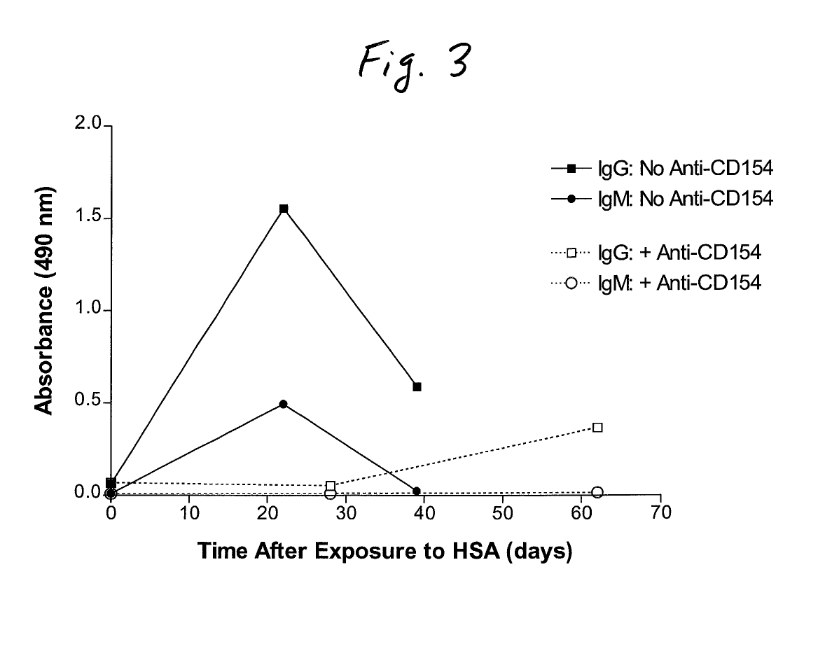

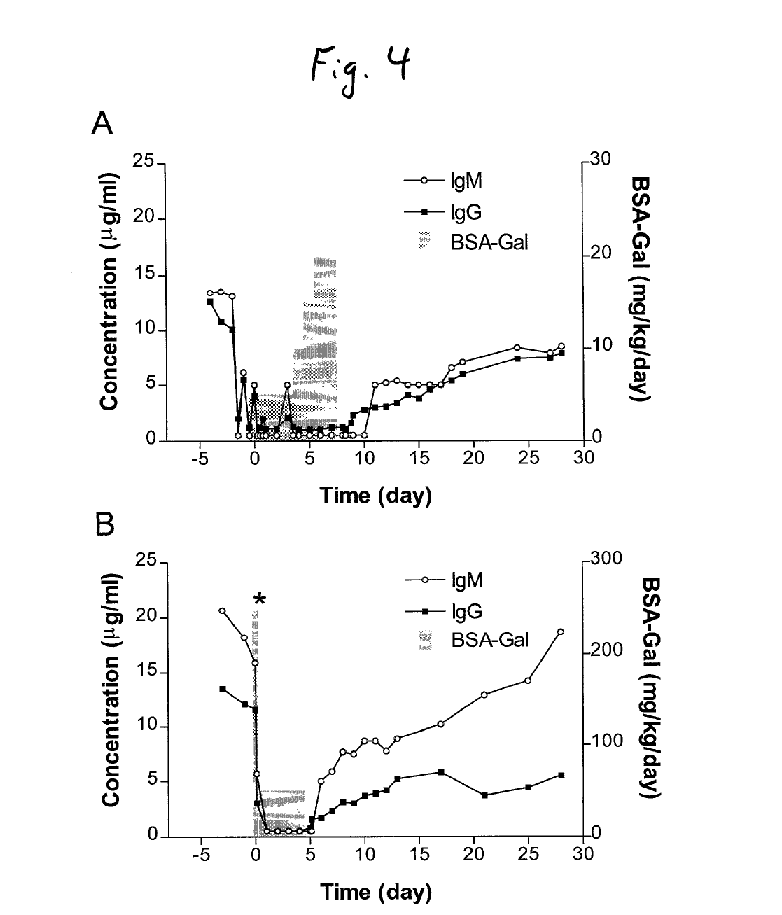

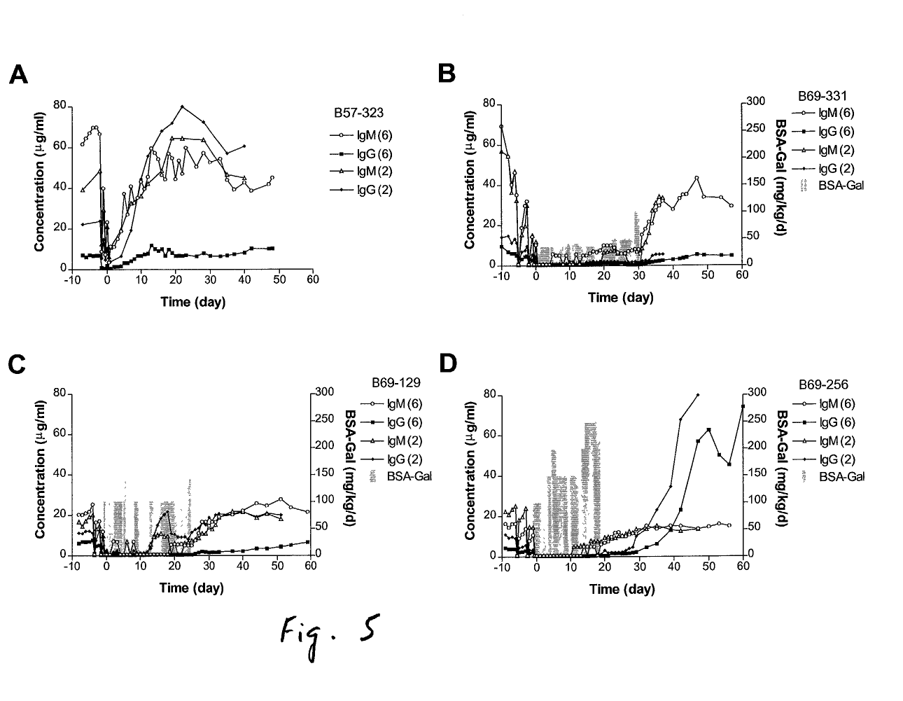

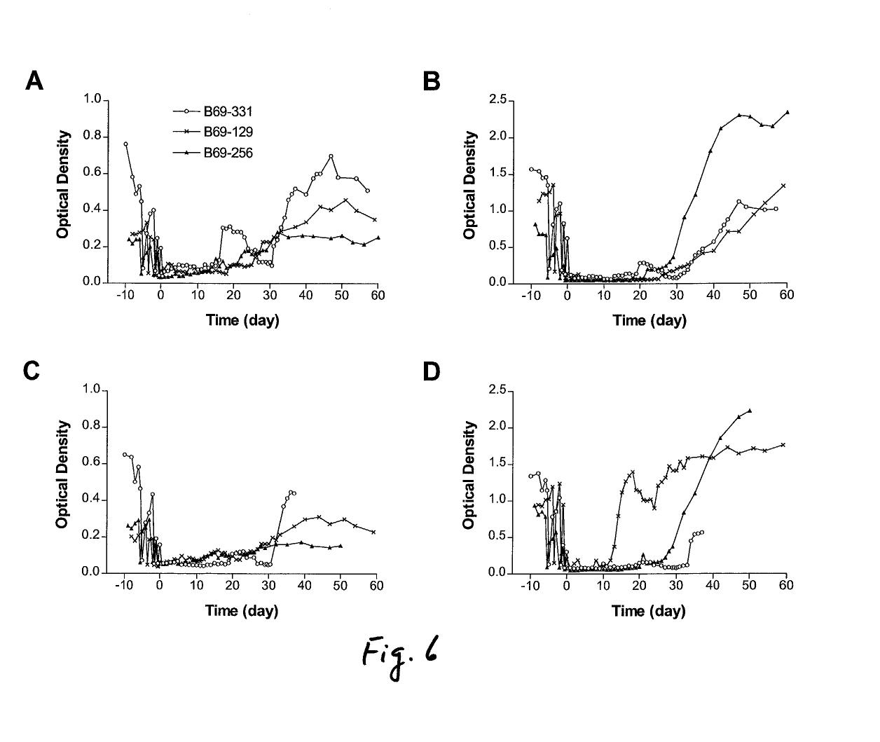

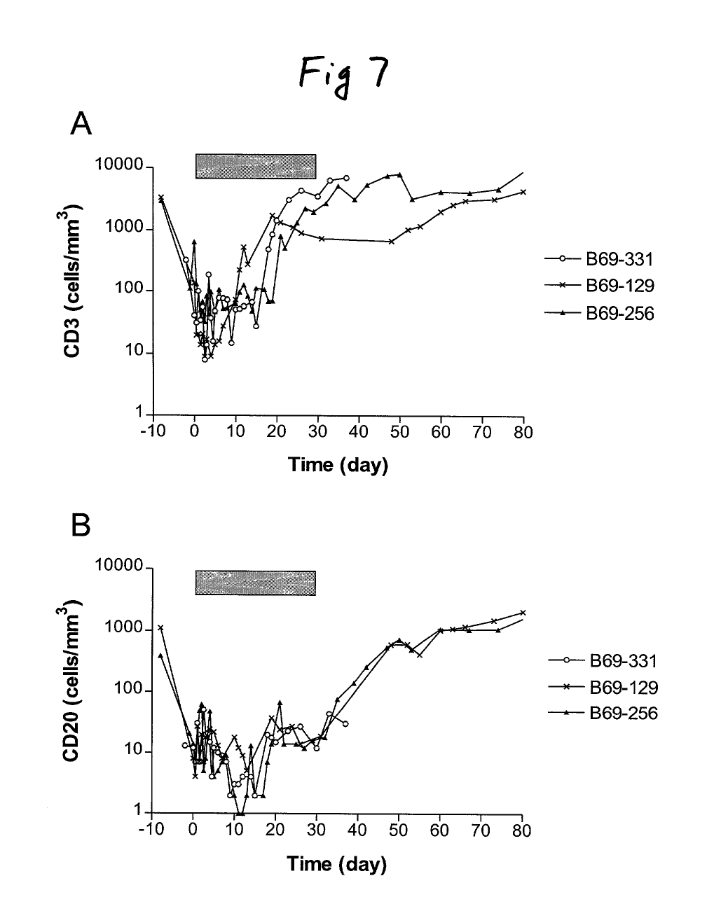

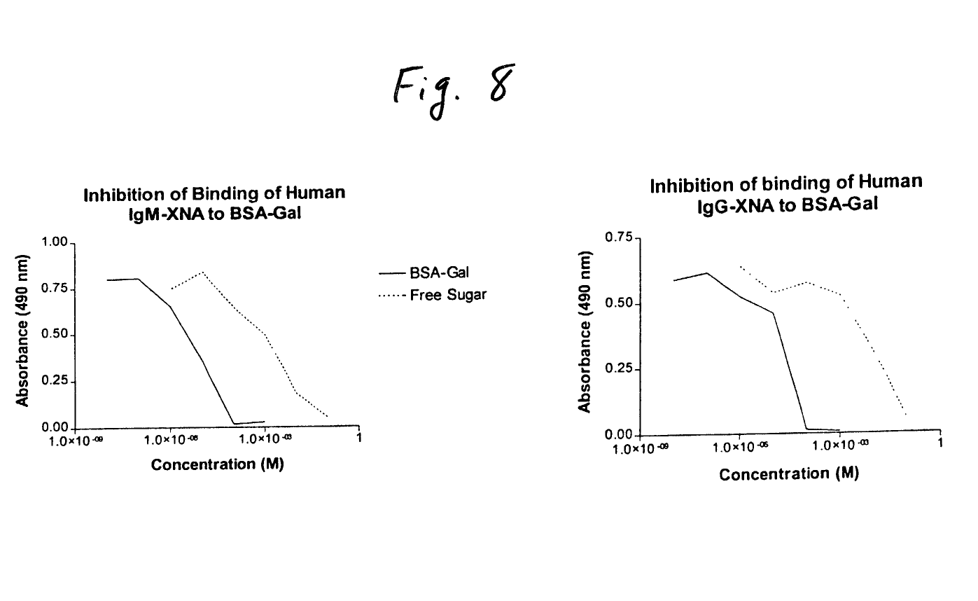

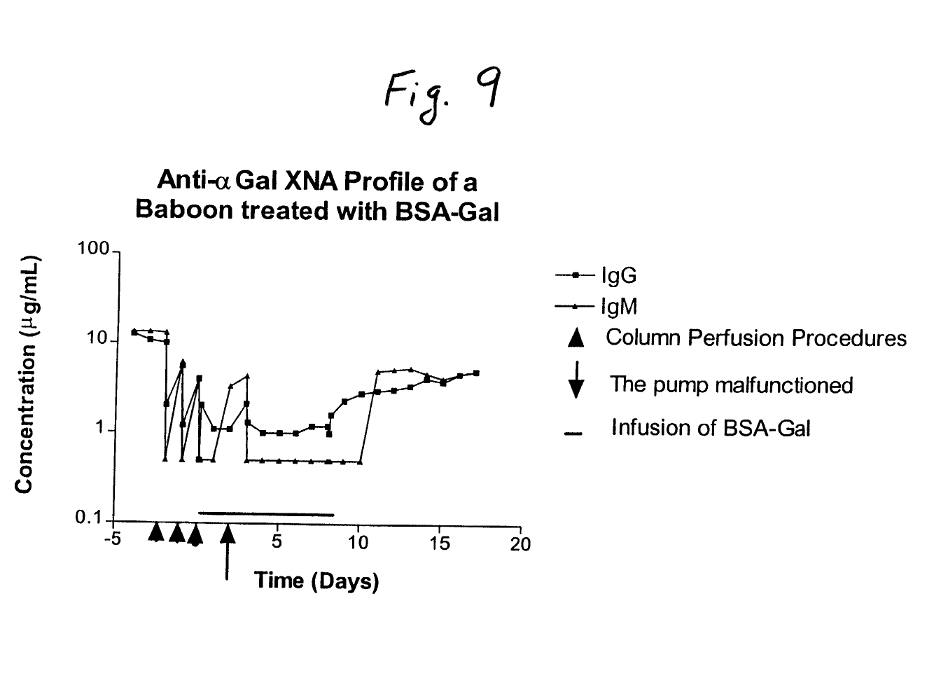

All recipient baboons underwent splenectomy on day −8, nonmyeloablative whole body irradiation in two fractions (150 cGy each) on days −6 and −5 (total dose 300 cGy), thymic irradiation (700 cGy) on day −1, horse anti-human antithymocyte globulin (ATGAM, Upjohn, Kalamazoo, Mich.) 50 mg/kg/day i.v. on days −3, −2 and −1, and EIA on days −6, −3 and −1 (FIG. 2) (12,25). This was followed by porcine PBPC transplantation on day 0 and further PBPC transplants on days 1 and 2. All baboons also received mycophenolate mofetil (donated by Roche, Nutley, N.J.) by continuous i.v. infusion (at 80-140 mg/kg/day, administered with an Abbott Omniflow 4000 infusion device) beginning on day −8 to maintain a whole blood level of 3-6 μg/ml (32). All baboons also received cobra venom factor i.v. at approximately 35-105 units/kg/day on days −1 to 14 or 28 to maintain the CH50 at 0% (33), and murine anti-human CD154 mAb (5C8; ATCC, Rockville, Md.) (20 mg/kg/day i.v.) administered on alternate days (13). In all but 2 experiments (Table 3), CyA (donated by Novartis, Basel, Switzerland) (at approximately 15 mg/kg/day) was administered by continuous i.v. infusion from days −8 to 28 to maintain a whole blood level of 1200-1400 ng/ml.

### BSA-Gal Therapy

Bovine serum albumin conjugated to Gal oligosaccharides (BSA-Gal) was provided by the ARC. It was diluted in saline, and administered either as a bolus i.v. infusion of 50 mg/kg over 2 hours or by a continuous i.v. infusion at rates varying from 20-250 mg/kg/day.

### Porcine PBPC Transplantation and Hematopoietic Growth Factor Therapy in Baboons

These methods rely on known protocols (13). The total number of PBPCs administered to each baboon was 2-4×10¹⁰ cells/kg. As the infusion of high doses of porcine PBPC was found to lead to features of a thrombotic microangiopathic state in the recipient baboons (17,34), all baboons received therapy aimed at preventing such activation. This consisted of prostacyclin (PGI2; 20 ng/kg/min by continuous i.v. infusion), heparin (10 U/kg/h by continuous i.v. infusion), and methylprednisolone (2 mg/kg×2 daily i.v. for 7 days, followed by tapering and discontinuation over the next 7 or 21 days). This therapy was begun immediately before the first PBPC infusion and continued for 14 or 28 days. Porcine interleukin- 3 (100-400 μg/kg/day s.c. or i.v.) and porcine stem cell factor (100-2000 μg/kg/day s.c. or i.v.) (35,36) were administered to all baboons from days 0 to 14 or 28.

### Assays for Detection of Baboon Anti-Gal Ab and Detection of Emergence of Ab Directed Against Porcine Non-Gal Determinants

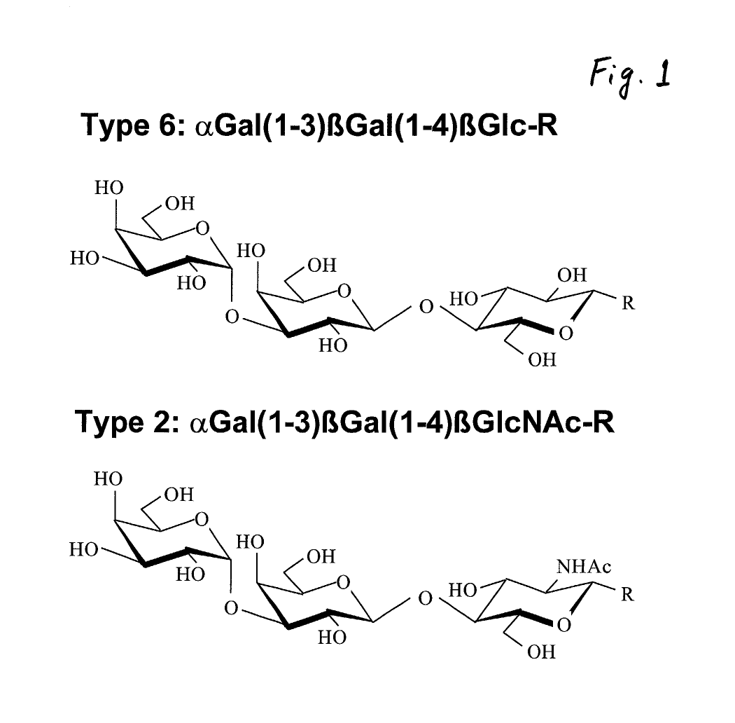

These methods rely on known protocols (9,25). IgM and IgG Ab reactive with Gal type 6 and type 2 (FIG. 1) were determined by ELISA, and anti-pig IgM and IgG were determined by flow cytometric analysis (FACS).

### Measurement of Serum Levels of Anti-CD154 mAb (ELISA)

These are known protocols (13).

### Measurement of Serum Levels of BSA-Gal

A competitive inhibition of binding ELISA was used to determine the levels of BSA-Gal in baboon serum samples. Briefly, sera collected pre-BSA-Gal infusion and at different times post-infusion were mixed with biotinylated BSA (Amresco, Solon, Ohio) and loaded on to plates coated with rabbit anti-BSA Ab (ICN Biomedicals, Los Angeles, Calif.). The higher the level of BSA-Gal in the serum sample, then the lower was the binding to the biotinylated BSA on the plate and, consequently, the lower the signal detected following color development by streptavidin-horseradish peroxidase and ABTS (2,2′-azino-di(3-ethyl)benzthiazoline-sulfonate) (Kirkegaard & Perry, Baltimore, Md.) as a substrate. Quantification was achieved by intrapolation against a standard curve generated by graded doses of BSA-Gal.

### Measurement of Antibodies to BSA-Gal

Measurement of anti-BSA Ab consisted of loading serum samples on to plates coated with BSA. Bound Ab was detected by donkey anti-human IgG Ab (Accurate, Westbury, N.Y.) and rabbit anti-human IgM (Dako, Copenhagen, Denmark) conjugated to horseradish peroxidase (HRP). Color development was achieved by ABTS as a substrate.

### Assay of Serum Cytotoxicity to Pig Cells

This complement-mediated cytotoxicity assay is based on a known protocoly (9). Results were expressed as cytotoxicity index, which was calculated as the inverse of the serum dilution that caused 50% killing of pig cells.

### FACS for Detection of Baboon T and B Cells

Flow cytometry to detect T and B cells was performed on blood, bone marrow (aspirates obtained from the iliac crests), and lymph nodes (biopsies obtained from either inguinal or axillary regions). The direct conjugated Ab anti-CD3 FITC (Biosource, Amarillo, Calif.) and anti-CD2 PE (Leu-5b, Becton Dickinson) were used as T cell markers, and anti-CD20 FITC (Leu-16, Becton Dickinson) and anti-CD22 PE (Clone RFB4, Caltag Laboratories, Burlingame, Calif.) as B cell markers. Blood and bone marrow were incubated at 4° C., lysed at room temperature with ACK-lysing buffer (Bio-Whittaker, Walkersville, Md.), washed and resuspended in 500 μl FACS Medium (1% BSA and 0.1% azide in phosphate-buffered saline (PBS)). Lymph nodes were mashed, filtered, and resuspended in 500 μl FACS medium. Cell count was approximately 1×10⁶/100 μl in all samples. The samples were stained using the aforementioned T and B cell-specific Ab. Acquisition was performed under hi-flow using the FACScan (Becton Dickinson), and samples were analyzed using WinList (Verity Software House, Topsham, Me.).

### Production ELISPOT for Detection of Anti-Gal Ab and Total Immunoglobulin

For detection of anti-Gal Ab production, 96-well MultiScreen-HA plates (MAHAS 4510 mixed cellulose esters, Millipore, Bedford, Mass.) were coated with 100 μl/well (5 μg/ml in PBS) of Gal-BSA or control BSA (ARC) at 4° C. overnight. For detection of total IgM or IgG production, goat anti-human IgM or IgG (Southern Biotech, Birmingham, Ala.) were used as coating reagents with goat anti-mouse IgM or IgG as negative controls. Plates were washed with PBS and blocked with IMDM and 0.4% BSA for 1 hour at 37° C. A total of 1×10⁶ cells/well, with a serial 5-fold dilution, were cultured overnight in a modified hybridoma medium (10% fetal calf serum was replaced by 0.4% BSA) at 37° C. with 5% CO₂. The plates were then washed with PBS and PBS-Tween (0.1% Tween-20). Ab production was detected with 100 μl goat anti-human IgM or IgG conjugated to HRP (Southern Biotech) at 1:1000 dilution in PBS with 1% BSA and 0.5% Tween at 4° C. for 1 hour. Plates were washed with PBS-Tween and PBS. Spots were visualized with substrate AEC or 4CN (Sigma) under a stereomicroscope (Nikon SMZ-U) equipped with a vertical white light. Data were presented as spot-forming units (SFU) per 10⁶ cells.

### FACS and Polymerase Chain Reaction for Detection of Pig Cell Chimerism in the Blood

These techniques rely on known protocols (13,37).

### Monitoring for Toxicity and Supportive Therapy

Blood cell counts, chemistry, coagulation parameters, and levels of immunosuppressive drugs were determined by routine methods. If the hematocrit fell <20%, washed irradiated red blood cells from ABO-matched baboon donors were administered. Erythropoietin was also administered to some baboons at a dose of 100 units/kg s.c. or i.v. Thrombocytopenia of <10,000 platelets/mm³ was corrected by the transfusion of fresh washed and irradiated baboon platelets. All pigs and baboons received daily cefazolin sodium (500-1000 mg/day i.v.) throughout the periods of leukapheresis and therapy, respectively. Blood cultures were monitored twice weekly and antibiotic therapy modified, if indicated.

### EXAMPLE 1

### Inhibition of Antibody Binding by BSA-Gal

The ability of BSA-Gal, as described herein, to inhibit binding of human serum (natural antibodies) to plates coated with BSA-Gal and pig cells. Results are depicted graphically in FIG. 8 and the data collected is shown in Table 1. Here, reactivity of pig cells of serum samples collected at different times from the same baboon or from different baboons before or after adding 20 mg/mL BSA-Gal was measured by FACS analysis as described herein. Bound antibodies were detected by fluorescent anti-human IgG and IgM antibodies. Results are expressed as median fluorescence intensity. There was a significant drop in anti-pig reactivity was observed after adding BSA-Gal.

TABLE 1

| Reactivity With Pig Cells | |
|---|---|
| (Median Fluorescence Activity) | |
| IgM Competition | IgG Competition |

| Sample | Pre | Post | Pre | Post |
|---|---|---|---|---|
| 1 | 257.1 | 66.1 | 28.9 | 14.6 |
| 2 | 112.4 | 62.6 | 47.3 | 16.6 |
| 3 | 165.5 | 61.5 | 19.8 | 13.3 |
| 4 | 199.9 | 63.8 | 58.8 | 17.9 |
| Negative Control | 24 | NA | 13.3 | NA |

### EXAMPLE 2

### In Vivo Removal of Anti-Gal Antibodies from the Circulation of Baboons

a baboon was treated with an escalating dose of Gal-BSA: on days 0-3 the dose was 5 mg/kg/day, on days 4 and 5 the dose was 10 mg/kg/day, on days 6 and 7 the dose was 10 mg/kg/day, and the last dose was on day 8 at 20 mg/kg/day. On day 0 the baboon received 2 doses of 20 mg/kg anti-CD154, thereafter on days 2, 4, 6, 8,10, 12, and 14 the baboon received one dose of 20 mg/kg. The baboon was also treated with mycophenolate mofetil (Roche Laboratories, Nutley, N.J.) at a dose of 100 mg/kg/day from day 0 through day 14 (see Table 2). The natural antibody profile is depicted in FIG. 9. Serum samples were collected on the designated days from baboons that were infused with human serum albumin (HSA) and either treated (B68-54 and B69-169) or not (B133-69 and B68-19) with anti-CD154 were assessed for reactivity against HSA in an ELISA assay. Bound antibodies were detected by HRP conjugated anti-human IgG and IgM. Baboons treated with anti-CD154 either did not generate anti-HSA antibodies or developed them later after anti-CD154 cleared from the circulation. In contrast, baboons that were not treated with anti-CD154 developed within 3-4 weeks a substantial response against HSA.

TABLE 2
B68-19 (Absorbence, 490 nm)

| Concentra- | | IgG | | | IgM | | |
|---|---|---|---|---|---|---|---|
| tion (%) | Day No. | 13 | 35 | 52 | 13 | 35 | 52 |
| 2 | | 0.063 | 1.556 | 0.589 | 0.011 | 0.494 | 0.024 |
| 0.4 | | 0.019 | 0.726 | 0.204 | 0.003 | 0.177 | 0.006 |
| 0.08 | | 0.007 | 0.183 | 0.0460 | 0 | 0.047 | 0 |
| 0.016 | | 0.014 | 0.04 | 0.01 | 0 | 0.011 | 0 |

B133-69 (Absorbence, 490 nm)

| Concentra- | | IgG | | | IgM | | |
|---|---|---|---|---|---|---|---|
| tion (%) | Day No. | 8 | 21 | 58 | 8 | 21 | 58 |
| 2 | | 0.05 | 0.846 | 1.707 | 0.021 | 0.115 | 0.124 |
| 0.4 | | 0.018 | 0.293 | 1.206 | 0.005 | 0.027 | 0.029 |
| 0.08 | | 0.011 | 0.071 | 0.441 | 0.001 | 0.006 | 0.005 |
| 0.016 | | 0.025 | 0.007 | 0.173 | 0.001 | 0.00 | 0.004 |

B68-54 (Absorbence, 490 nm)

| Concentra- | | IgC | | | IgM | | |
|---|---|---|---|---|---|---|---|
| tion (%) | Day No. | −6 | 22 | 56 | −6 | 22 | 56 |
| 2 | | 0.068 | 0.053 | 0.366 | 0.008 | 0.010 | 0.015 |
| 0.4 | | 0.019 | 0.009 | 0.095 | 0.002 | 0.002 | 0.004 |
| 0.08 | | 0.007 | −0.004 | 0.012 | 0.00 | 0.00 | 0.00 |
| 0.016 | | 0.011 | −0.005 | 0.003 | 0.00 | 0.00 | 0.00 |

B69-169 (Absorbence, 490 nm)

| Concentra- | | IgG | | | IgM | | |
|---|---|---|---|---|---|---|---|
| tion (%) | Day No. | −3 | 24 | 54 | −3 | 24 | 54 |
| 2 | | 0.049 | 0.017 | 0.041 | 0.009 | 0.017 | 0.02 |
| 0.4 | | 0.015 | 0.002 | 0.008 | 0.002 | 0.004 | 0.005 |
| 0.08 | | 0.006 | −0.005 | −0.004 | 0.00 | 0.00 | 0.00 |
| 0.016 | | 0.005 | −0.01 | −0.005 | 0.001 | 0.00 | 0.00 |

### EXAMPLE 3

### Use of BSA-Gal in Baboons

In vitro inhibition of anti-Gal Ab binding to pig cells by BSA-Gal The binding of baboon sera to pig peripheral blood mononuclear cells was assessed by FACS, and compared with binding to the same sera after the addition of 20 mg/ml BSA-Gal. Bound anti-Gal Ab were detected by fluoresceinated anti-human IgM and IgG Ab, and the results were expressed as median fluorescence intensity.

### Prevention of Sensitization to Human Serum Albumin (HSA) by Anti-CD154 mAb Therapy in Baboons

Using stored baboon sera from previous studies in our laboratory, in which baboons had been administered HSA either when receiving anti-CD154 mAb therapy or not, anti-HSA Ab responses were measured using a modification of the ELISA for BSA described above.

### Response to i.v. Infusion of BSA-Gal in Baboons

One baboon underwent a course of EIA to deplete the existing anti-Gal Ab, followed immediately by the initiation of a continuous i.v. infusion of BSA-Gal at 5-20 mg/kg/day for 8 days. A second baboon was initially administered an i.v. bolus of BSA-Gal (50 mg/kg over 2 hours) followed by a continuous i.v. infusion for 5 days at 50 mg/kg/day. Kidney and liver biopsies were taken in this latter baboon on day 9 to search for abnormalities that might have been associated with toxicity from the high dose of the BSA-Gal administered. Both baboons also received anti-CD154 mAb (20 mg/kg i.v.) on alternate days to prevent sensitization to BSA-Gal. Both baboons had been exposed to PBPC some months previously, but anti-Gal IgM and IgG levels had been stable for several weeks.

### Experimental Groups (Table 3)

Group 1 baboons (n=4) received the nonmyeloablative regimen outlined in Materials and Methods (FIG. 2). In 2 baboons, a course of 8 doses of anti-CD154 mAb was administered without CyA, and in 2 a course of only 2 doses were combined with 28 days treatment with CyA.

TABLE 3
Summary of Therapy

| | | Basic | | | BSA-Gal |
|---|---|---|---|---|---|
| | Baboon | Therapy | | CD154 mAb | (dose range) |
| Group | #/wt (kg) | (a) | CyA | (# of doses) | (mg/kg/day) |
| 1 | B57-323 (12) | + | − | +(8) | − |
| | B68-54 (10) | + | − | +(8) | − |
| | B57-16 (13) | + | + | +(2) | − |
| | B57-301 (15) | + | + | +(2) | − |
| 2 | B69-331 (10) | + | + | +(23) | +(20 − 100) |
| | B69-129 (8) | + | + | +(19) | +(50 − 150) |
| | B69-256 (12) | + | + | +(16) | +(100 − 250) |

Basic therapy included splenectomy (day −8), whole body irradiation (150 cGy on days −6 and −5), thymic irradiation (700 cGy on day −1), extracorporeal immunoadsorption (days −6, −3 and −1), and immunosuppressive therapy with mycophenolate mofetil and anti-thymocyte globulin

Group 2 baboons (n=3) received the Group 1 regimen (including CyA). In addition, they received BSA-Gal by continuous i.v. infusion beginning immediately after the final EIA and continued for 19 to 30 days at rates between 20 to 250 mg/kg/day. In the initial baboon (B69-331), BSA-Gal was given at relatively low dose, but the dose was increased in the 2 subsequent experiments. To prevent sensitization to BSA-Gal, anti-CD154 mAb therapy was continued throughout the period of BSA-Gal infusion and then for a further 7 days after discontinuation of BSA-Gal. The period of anti-CD154 mAb therapy, therefore, extended from 29 to 43 days (16 to 23 doses).

### EXAMPLE 4

### Intravenous Infusion of BSA-Gal in Baboons

### In vitro Inhibition of Anti-Gal Ab Binding to Pig Cells by BSA-Gal

The addition of BSA-Gal to baboon sera reduced binding of anti-Gal IgM and IgG to pig cells at levels close to background (Table 4).

### Prevention of Sensitization to Human Serum Albumin (HSA) by Anti-CD154 mAb Therapy in Baboons

The i.v. infusion into the baboon of HSA in the absence of anti-CD154 mAb therapy resulted in generation of anti-HSA IgM and IgG within 20 days (FIG. 3). Exposure to HSA while receiving anti-CD154 mAb therapy did not result in the generation of anti-HSA Ab, although there was a small increase in anti-HSA IgG after discontinuation of the mAb (FIG. 3).

### Response to i.v. Infusion of BSA-Gal in Baboons

In the first baboon, EIA was successful in depleting all IgM and IgG reactive to Gal type 6 and type 2 (not shown), although some recovery of IgG had occurred within 18 hours of the last EIA (FIG. 4A). The initial i.v. infusion rate of BSA-Gal (5 mg/kg/day) proved inadequate, but an increase to 10 and, later, 20 mg/kg/day was successful in maintaining both Gal-reactive IgM and IgG at very low levels. When BSA-Gal was discontinued (day 8), IgM remained undetectable for a further 48 hours although IgG started to recover within 24 hours. The levels never returned to pre-EIA levels.

In the second baboon, the bolus of BSA-Gal (50 mg/kg) resulted in a dramatic reduction of IgM and IgG reactive to both Gal type 6 and type 2 (not shown) to unmeasurable levels (FIG. 4B). The continuous i.v. infusion of BSA-Gal at 50 mg/kg/day maintained undetectable levels until the infusion was discontinued after 5 days (on day 4). Even 3 weeks later, the level of anti-Gal IgG remained at approximately 30% of the pretreatment level.

In neither experiment were there any abnormalities in blood chemistry to indicate any immune complex formation or any toxicity associated with BSA-Gal infusion. However, a liver biopsy taken 5 days after initiation of BSA-Gal therapy in the baboon that received the bolus injection of BSA-Gal showed some hepatocyte swelling (not shown). A kidney biopsy taken at the same time was normal.

### Experimental Groups

### Effect of Nonmyeloablative Regimen on Hematopoietic Parameters

The results in Group 1 have been reported previously (13). The nonmyeloablative regimen was well-tolerated in baboons of both Groups, although there were significant reductions in white blood cell (to <2,000 /mm³) and platelet (to <20,000 /mm³) counts (not shown). Maximum suppression of white blood cell count was at approximately day 12 in both groups, and platelet levels were lowest on day 9. It was our impression that the Group 2 baboons maintained better appetite and activity throughout the leukopenic period, even though the degree and period of leukopenia was marginally greater in this group.

In both Groups, after 2 doses of anti-CD154 mAb, serum levels reached 200-300 μg/ml, peaking to 300-500 μg/ml after subsequent doses (data not shown) (13). After discontinuation of the mAb, the serum level fell steadily, with a half-life of approximately 2 days, becoming undetectable within 14 days.

TABLE 4
Binding Of Baboon Sera To Pig Cells Before And After The
In Vitro Addition Of BSA-Gal (20 mg/ml)

| Reactivity with Pig Cells (MFI) | |
|---|---|
| IgM competition | IgG competition |

| Serum | Pre | Post | Pre | Post |
|---|---|---|---|---|
| 1 | 257 | 66 | 29 | 15 |
| 2 | 112 | 63 | 47 | 17 |
| 3 | 166 | 62 | 20 | 13 |
| 4 | 200 | 64 | 59 | 18 |
| Negative control | 24 | NA | 12 | NA |

MFI = median fluorescence intensity (measured by FACS)
NA = not applicable

### Antibody Responses to Porcine PBPC Transplantation

Group 1: In the 2 baboons that received a course of 8 doses of anti-CD154 mAb without CyA, although anti-Gal Ab gradually returned to baseline level, no or minimal increase above pre-PBPC (PBPC=mobilized porcine peripheral blood progenitor cells) levels was detected (Table 5 and FIG. 5A) (with follow-up for a minimal of 3 months). In the 2 baboons in which anti-CD154 mAb was discontinued after 2 doses but CyA continued for 28 days, a late (at 30 days) anti-Gal Ab response was seen in both baboons (not shown) (13). IgM showed no increase in one baboon and a 4-fold increase in the other; IgG showed an 8-30-fold increase. No Ab against porcine non-Gal determinants was detected in any baboon.

Group 2: Anti-Gal Ab remained undetectable or at minimally-detectable levels while BSA-Gal was being administered (FIGS. 5B and 6), although several increases in the rate of infusion were required in each baboon to obtain this result. In one baboon (B69-129), Ab to Gal type 2 returned even when Ab to Gal type 6 remained undetectable (FIGS. 5C and 6). This was the only baboon in which there was a lack of correlation between the levels of these two Ab. Unexpectedly, BSA-Gal (which is made up of Gal type 6) failed to maintain depletion of anti-Gal Ab reactive with Gal type 2 even though prior EIA (performed with an immunoaffinity column of Gal type 6) was successful in removing anti-Gal Ab reactive with both Gal type 6 and Gal type 2 (FIG. 5C). A limitation on availability of BSA-Gal prevented longer periods of administration in each baboon. After discontinuation of both BSA-Gal and anti-CD154 mAb in one baboon (B69-256), induced anti-Gal IgG appeared with the level rising 15-fold compared to the baseline level (FIGS. 5D and 6). This was believed to be due to the continuing presence of a high level of pig cell microchimerism after cessation of anti-CD154 mAb therapy. No Ab against porcine non-Gal determinants was detected in any baboon.

FIG. 6 illustrates the changes in optical density in all 3 Group 2 baboons.

As the lower limit of detection by ELISA was 5 μg/ml for IgM and 0.5 μg/ml for IgG, the optical density to some extent provides a clearer indication of the effectiveness of the BSA-Gal therapy.

Whereas the levels of circulatory BSA-Gal in two baboons reached a maximum of >3 mg/ml, the level in B69-129 did not exceed 0.5 mg/ml (for unknown reason).

TABLE 5
Changes In Anti-αGal IgM And IgG And Development Of
Antibodies To New Pig Antigenic Determinants Following
Porcine Pbpc Transplantation In Baboons

| | | Return of Natural | | | |
|---|---|---|---|---|---|
| Group | | Anti-Gal Antibody | | | Sensitization to |
| Determinants | Baboon | Type 6 | Type 2 | Gal | Porcine Non-Gal |
| 1 | B57-323 | + | + | − | − |
| | B68-54 | + | + | − | − |
| | B57-16 | + | − | + (a) | − |
| | B57-301 | + | + | + (a) | − |
| 2 | B69-331 | − | − | − | − |
| | B69-129 | − | + | − | − |
| | B69-256 | − | − | + (b) | − |

(a) Late and attenuated increase in anti-Gal Ab (see text).
(b) Associated with a continuing high level of pig cell microchimerism after cessation of anti-CD154 mAb therapy.

### Serum Cytotoxicity to Pig Cells (Table 6)

Serum anti-pig cytotoxicity remained negligible or low in all baboons throughout the period of administration of cobra venom factor (data not shown). If rabbit complement were added to the serum in vitro, cytotoxicity to pig cells could clearly be demonstrated whenever anti-Gal Ab was measurable.

In the Group 1 baboons, by day 20, cytotoxicity remained unchanged or lower compared to pre-transplant (Table 6), correlating with a slow return of anti-Gal Ab, which had not yet reached baseline level in some cases. In the two Group 1 baboons in which CyA was discontinued on day 28, cytotoxicity rose 8-fold in comparison with the continued baseline level in the other two baboons in this group (Table 6). This increase in cytotoxicity correlated with an increase in the level of anti-Gal Ab.

In Group 2, cytotoxicity remained negligible while anti-Gal Ab remained undetectable, but increased with return of Ab after discontinuation of BSA-Gal therapy (not shown). In B69-129, in which Ab reactive with Gal type 2 was detectable in the absence of Ab to Gal type 6, cytotoxicity became detectable while BSA-Gal therapy was continuing (Table 6).

### Toxicity of BSA-Gal Infusion

No toxic effects of BSA-Gal administration were observed in any of the 3 baboons to which it was administered. A comparison of parameters of renal and hepatic function indicated no significant differences between the baboons of Groups 1 and 2.

TABLE 6
Serum Cytotoxicity (Mean Index) To Pig Cells (a)

| | | Post-PBPC | Post-PBPC |
|---|---|---|---|
| Group | Pre-PBPC | (Days 20-30) (b) | (Days > 40) (b) |
| 1 (c) | 1 | 0.8 | 0.8 |
| (d) | 1 | 0.3 | 6.0 |
| 2 (B69-331) | 1 | 0.1 | 0.1 |
| (B69-129) | 1 | 0.3 | 0.5 (e) |
| (B69-256) | 1 | 0.3 | 1.1 |

(a) This in vitro serum cytotoxicity assay has been fully described previously (6). Rabbit complement was added to all serum samples. Mean cytotoxicity index is calculated as the inverse of the serum dilution that causes 50% killing of pig cells divided by the pre-PBPC value (to give a pre-PBPC index of 1).
(b) In baboons undergoing the same nonmyeloablative regimen but not receiving anti-CD154 mAb therapy, the serum cytotoxicity index is 5-7 at both of these time points (13).
(c) Anti-CD154 mAb × 8 doses, no CyA.
(d) Anti-CD154 mAb × 2 doses, CyA for 28 days.
(e) Associated with return of Ab directed against Gal type 2.

### FACS for Detection of Baboon T and B Cells

In Group 1, though CD20 cells were not measured, CD3 cells had recovered to baseline within approximately 14-21 days (not shown). In Group 2, following WBI (days −6 and −5) and the course of antithymocyte globulin (days −3, −2, -1), CD3 cells were largely depleted (FIG. 7A) but had recovered to baseline levels before the course of BSA-Gal therapy had been completed between days 19-30. CD20 cells were more efficiently depleted by the induction therapy (FIG. 7B), and in 2 baboons their numbers remained low until BSA-Gal was discontinued, after which there was a more rapid increase. In these 2 baboons, the number of CD20 cells did not return to baseline until approximately day 50.

### ELISPOT for Detection of Anti-Gal Ab and Total Immunoglobulin Production

The number of antibody secreting cells in baboon lymph nodes and bone marrow remained relatively stable throughout the study (data not shown), indicating that B cell tolerance to Gal did not develop.

### FACS and Polymerase Chain Reaction for Detection of Pig Cell Chimerism in the Blood

Group 1: Pig cell macrochimerism (detectable by FACS) was detected for 5 days, with maxima of 33% and 73% when CyA was omitted or included in the regimen, respectively (37). Only one baboon showed reappearance of pig cells subsequently by FACS. In B57-16, presumed cell fragments (based on low forward-side scatter property) staining positive with a pan-pig Ab were detected from days 9 to 16. Pig monocytes were documented on days 16 to 22, with a maximum of 6% on day 19 (not shown). Microchimerism, however, was continuously present in the blood of all 4 baboons for a maximum of 33 days, and thereafter was intermittently detected for >100 days (37).

Group 2: Macrochimerism reached a maximum of 36% while PBPC were being infused, after which it was lost in one baboon. In the other two baboons, macrochimerism was detected continuously at low levels (<1%) until day 14 and 17, respectively. In all 3 baboons, microchimerism (detectable by PCR) was continuous for a maximum of 32 days, and has been intermittent for >75 days to date. B69-256 maintained a relatively high level of microchimerism intermittently after discontinuation of anti-CD154 mAb therapy (not shown), which was thought to be the cause of the development of induced Gal-reactive Ab in this baboon (FIGS. 5D and 6).

In previous treatments (8,32), a course of EIA has been followed by a return of detectable Ab within 18 hours, usually increasing to pre-EIA levels within 3-5 days despite pharmacologic immunosuppressive therapy. Even repeated EIA (of up to 9 EIAs over 2-3 weeks) did not prevent return of Ab from beginning within hours (32). Although anti-CD154 mAb therapy prevented an induced Ab response, it had no effect on the return of natural Ab (13). The present invention is directed toward the ability of the continuous infusion of BSA-Gal to maintain the depletion of Ab following EIA.

The data of Example 3 demonstrate that (i) the addition of BSA-Gal to baboon serum in vitro resulted in near complete depletion of anti-Gal Ab reactivity to pig cells, (ii) anti-CD154 mAb therapy prevented the development of induced Ab to HSA in baboons, and (iii) BSA-Gal could maintain the in vivo depletion of anti-Gal Ab brought about by EIA or, alternatively, if administered as a bolus, could deplete Ab without the need for EIA.

Comparison of the return of Ab after EIA between Groups 1 and 2 indicated that the continuous i.v. infusion of BSA-Gal was largely successful in maintaining depletion of Ab, which is in contrast to previous results (8,10,14,25). Because of the much lower natural levels of Gal-reactive IgG than IgM in baboons, it was easier to maintain depletion of IgG. Furthermore, the administration of BSA-Gal at the dosages given appeared to be non-toxic and without side effects. No evidence was seen of immune complex formation by laboratory tests of renal or hepatic dysfunction. After bolus therapy, however, although there were no biochemical features of renal or hepatic dysfunction, a liver biopsy taken 5 days after initiation of therapy showed some hepatocyte swelling (not shown). A kidney biopsy taken at the same time, however, was normal.

In earlier protocols, when high numbers of PBPC were infused (2-4×10¹⁰ cells/kg) into baboons following a nonmyeloablative regimen similar to that outlined in the current studies, sensitization - defined as an increase in Gal-reactive Ab above the baseline (pre-PBPC) level and/or the development of Ab to new pig (non-Gal) determinants—clearly developed, and was associated with a 7-fold rise in the serum cytotoxicity index (13). The inclusion of an anti-CD154 mAb in the protocol (as in the Group 1 baboons in the present study) completely or largely inhibited the development of sensitization, and was associated with maintenance of a low serum cytotoxicity index. When the course of anti-CD154 mAb consisted of only 2 doses, and CyA was administered for 28 days, late attenuated sensitization to Gal, but not to porcine non-Gal determinants, occurred after CyA was discontinued. This late induced Ab response was considerably less than that seen previously in sensitized baboons not receiving anti-CD154 mAb therapy. The anti-Gal IgG response was 8-30-fold in the Group 1 baboons, whereas it was >100-fold in baboons where anti-CD154 mAb therapy had not been administered (13). As the level of chimerism observed appeared higher in the Group I baboons when CyA was retained in the regimen (37), CyA therapy was combined with a prolonged course of anti-CD154 mAb in Group 2. This prolonged course indeed prevented even an attenuated induced anti-Gal Ab response and also had the desired effect of preventing sensitization to BSA-Gal.

The beneficial effects of anti-CD154 mAb in this model have been fully discussed previously (13). From our observations, we concluded that, although blockade of the CD40-CD154 pathway prevented an induced T cell-dependent humoral response, it had no effect on T cell-independent natural anti-pig Ab production. The present study demonstrates that BSA-Gal therapy goes some considerable way to resolve the problem of natural anti-Gal Ab, at least in the short-term. However, longer periods of therapy will be required to ensure that its beneficial effect can be maintained and that toxic side-effects do not develop. At present, we have been greatly restricted by its limited availability and high cost. We are currently assessing its effect on survival of pig kidney transplants in baboons.

The combination of BSA-Gal and anti-CD154 mAb, by preventing return of natural anti-Gal Ab and the induction of other anti-pig Ab, may provide a “window” during which pig hematopoietic cell engraftment can be achieved, leading to a state of immunological tolerance. Alternatively, this combination therapy may facilitate the development of a state of accommodation, if this is truly achievable in discordant xenotransplantation. Dalmasso et al. (38) have presented in vitro data that suggest that the presence of anti-pig IgM in the absence of complement activation results in accommodation of transplanted tissues. If high levels of anti-Gal IgG and non-Gal IgG do not develop following anti-CD154 mAb therapy, and IgM is allowed to return only slowly to a sub-pretransplant level, this may provide ideal conditions to explore whether accommodation develops.

Our observation that the rate of return of natural anti-Gal Ab following EIA was reduced when BSA-Gal had been administered than when EIA followed any other form of immunosuppressive therapy suggested that BSA-Gal may have had an inhibitory effect on the B/plasma cells producing the Ab, or even a partial tolerizing effect. The observation that recovery of CD20 cells appeared delayed during BSA-Gal therapy in 2 baboons (FIG. 6B) supports this conclusion. However, ELISPOT assay did not demonstrate any reduction in anti-Gal Ab production. Further investigation will be required to clarify these possibilities.

These data are in accord with previous observations by us and others that some polymorphism exists in Gal-reactive Ab (1,39-45). It has been demonstrated that Gal trisaccharides are more efficient in blocking Gal-reactive Ab than is the Gal disaccharide (41,42). Neethling et al (41) showed that the reducing end of Gal oligosaccharides contributed to their efficiency in blocking anti-Gal Ab, but did not compare Gal trisaccharide type 2 and type 6 in this respect. EIA has been performed by us in almost 100 baboons using a Gal trisaccharide type 6 immunoaffinity column (10). In a subgroup of these baboons (n=8), in which we have tested for the presence of Ab directed against the type 2 saccharide, the type 6 saccharide has always been successful in adsorbing type 2-reactive Ab as well as type 6-reactive Ab. However, in one of the baboons in the present study, Ab directed against the type 2 saccharide returned while BSA-Gal was being administered (FIGS. 5C and 6). In contrast, type 6-reactive Ab remained undetectable. The presence of type 2-reactive Ab was associated with a rise in the serum cytotoxicity index.

As the anti-Gal type 2 IgG level was high in this baboon before exposure to the PBPC, it is possible that this baboon had been sensitized to the type 2 saccharide in the past. How such sensitization develops in a baboon in the wild remains uncertain, but infection with a microorganism expressing the type 2 saccharide seems to be a possibility. Although the number of experiments we have performed using BSA-Gal to date is very few, our results suggest that Gal trisaccharide type 6 may be sufficient to adsorb all anti-Gal Ab in most baboons, but that the addition of the type 2 trisaccharide may be necessary in a few. This necessity could possibly be predicted by measuring levels of type 2-reactive Ab before PBPC transplantation.

Although the level of pig cell chimerism was low, two baboons in Group 2 were continuously positive by FACS for over 2 weeks. Nevertheless, the loss of macrochimerism lends support to our previous conclusion that factors other than Ab, such as macrophage activity, also play an important role in the survival of PBPC in a discordant host (46).

### References

1. Good A H, Cooper D K C, Malcolm A J, et al. Identification of carbohydrate structures that bind human antiporcine antibodies: implications for discordant xenografting in humans. Transplant Proc 1992; 24: 559.

2. Cooper DKC. Depletion of natural antibodies in non-human primates—A step towards successful discordant xenografting in humans. Clin Transplant 1992; 6:178.

3. Cooper D K C, Good A H, Koren E, et al. Identification of alpha-galactosyl and other carbohydrate epitopes that are bound by human anti-pig antibodies: relevance to discordant xenografting in man. Transpl Immunol 1993; 1: 198.

4. Sandrin M S, Vaughan H A, Dabkowski P L, McKenzie I F. Anti-pig IgM antibodies in human serum react predominantly with Gal(alpha 1-3)Gal epitopes. Proc Natl Acad Sci U S A 1993; 90: 11391.

5. Galili U. Interaction of the natural anti-Gal antibody with alpha-galactosyl epitopes: a major obstacle for xenotransplantation in humans. Immunol Today 1993; 14: 480.

6. Ye Y, Neethling F A, Niekrasz M, et al. Evidence that intravenously administered alpha-galactosyl carbohydrates reduce baboon serum cytotoxicity to pig kidney cells (PK15) and transplanted pig hearts. Transplantation 1994; 58: 330.

7. Taniguchi S, Neethling F A, Korchagina E Y, et al. In vivo immunoadsorption of antipig antibodies in baboons using a specific Gal(alpha)1-3Gal column. Transplantation 1996; 62: 1379.

8. Kozlowski T, lerino F L, Lambrigts D, et al. Depletion of anti-Gal(alpha)1-3Gal antibody in baboons by specific alpha-Gal immunoaffinity columns. Xenotransplantation 1998; 5: 122.

9. Xu Y, Lorf T, Sablinski T, et al. Removal of anti-porcine natural antibodies from human and nonhuman primate plasma in vitro and in vivo by a Galalpha1-3Galbeta1-4betaGlc-X immunoaffinity column. Transplantation 1998; 65:172.

10. Watts A, Foley A, Awwad M, et al. Plasma perfusion by apheresis through a Gal immunoaffinity column successfully depletes anti-Gal antibody: Experience with 320 aphereses in baboons. Xenotransplantation 2000; 7: 181.

11. Cooper D K C, Cairns T D H, Taube D H. Extracorporeal immunoadsorption of alphaGal antibodies. Xeno 1996; 4: 27.

12. Kozlowski T, Shimizu A, Lambrigts D, et al. Porcine kidney and heart transplantation in baboons undergoing a tolerance induction regimen and antibody adsorption. Transplantation 1999; 67: 18.

13. Buhler L, Awwad M, Basker M, et al. High-dose porcine hematopoietic cell transplantation combined with CD40 ligand blockade in baboons prevents an induced anti-pig humoral response. Transplantation 2000; 69: 2296.

14. Alwayn I P, Basker M, Buhler L, Cooper D K C. The problem of anti-pig antibodies in pig-to-primate xenografting: current and novel methods of depletion and/or suppression of production of anti-pig antibodies. Xenotransplantation 1999; 6: 157.

15. lerino F L, Kozlowski T, Siegel J B, et al. Disseminated intravascular coagulation in association with the delayed rejection of pig-to-baboon renal xenografts. Transplantation 1998; 66: 1439.

16. lerino F L, Gojo S, Banerjee P T, et al. Transfer of swine major histocompatibility complex class 11 genes into autologous bone marrow cells of baboons for the induction of tolerance across xenogeneic barriers. Transplantation 1999; 67: 1119.

17. Buhler L, Basker M, Alwayn I P J, et al. Coagulation and thrombotic disorders associated with pig organ and hematopoietic cell transplantation in nonhuman primates. Transplantation 2000; 70: 1323.

18. Buhler L, Yamada K, Alwayn I, et al. Miniature swine and hDAF pig kidney transplantation in baboons treated with a nonmyeloablative regimen and CD154 blockade. XVIII International Congress of The Transplantation Society 2000; 220 (Abstract 0618). Submitted for publication.

19. Lambrigts D, Sachs D H, Cooper D K C. Discordant organ xenotransplantation in primates: world experience and current status. Transplantation 1998; 66: 547.

20. Perper R J, Najarian J S. Experimental renal heterotransplantation. I. In widely divergent species. Transplantation 1966; 4: 377.

21. Linn B S, Jennsen J A, Portal P, Snyder G B. Renal xenograft prolongation by suppression of natural antibody. J Surg Res 1968; 8: 211.

22. Cooper D K C, Ye Y, Niekrasz M, et al. Specific intravenous carbohydrate therapy. A new concept in inhibiting antibody-mediated rejection-experience with ABO-incompatible cardiac allografting in the baboon. Transplantation 1993; 56: 769.

23. Simon P M, Neethling F A, Taniguchi S, et al. Intravenous infusion of Galalpha1-3Gal oligosaccharides in baboons delays hyperacute rejection of porcine heart xenografts. Transplantation 1998; 65: 346.

24. Romano E, Neethling F A, Nilsson K, et al. Intravenous synthetic alphaGal saccharides delay hyperacute rejection following pig-to-baboon heart transplantation. Xenotransplantation 1999; 6: 36.

25. Kozlowski T, Monroy R, Xu Y, et al. Anti-Gal (alpha)1-3Gal antibody response to porcine bone marrow in unmodified baboons and baboons conditioned for tolerance induction. Transplantation 1998; 66: 176.

26. Davies K A, Chapman P T, Norsworthy P J, et al. Clearance pathways of soluble immune complexes in the pig. Insights into the adaptive nature of antigen clearance in humans. J Immunol 1995; 155: 5760.

27. Davies K A, Hird V, Stewart S, et al. A study of in vivo immune complex formation and clearance in man. J Immunol 1990; 144: 4613.

28. Nardin A, Lindorfer M A, Taylor R P. How are immune complexes bound to the primate erythrocyte complement receptor transferred to acceptor phagocytic cells? Mol Immunol 1999; 36: 827.

29. Gonzalez M L, Waxman F J. Glomerular deposition of immune complexes made with IgG2a monoclonal antibodies. J Immunol 2000; 164: 1071.

30. Nash K, Chang Q, Watts A, et al. Peripheral blood progenitor cell mobilization and leukapheresis in pigs. Lab Anim Sci 1999; 49: 645.

31. Cooper D K C, Ye Y, Niekrasz M. Heart transplantation in primates. In: Handbook of Animal Model in Transplantation Research, Cramer D V, Podesta L G, Makowka L, eds. Boca Raton, CRC Press, 1994; 173

32. Lambrigts D, Van Calster P, Xu Y, et al. Pharmacologic immunosuppressive therapy and extracorporeal immunoadsorption in the suppression of anti-alphaGal antibody in the baboon. Xenotransplantation 1998; 5: 274.

33. Kobayashi T, Taniguchi S, Neethling F A, et al. Delayed xenograft rejection of pig-to-baboon cardiac transplants after cobra venom factor therapy. Transplantation 1997; 64: 1255.

34. Buhler L, Goepfert C, Kitamura H, et al. Porcine hematopoietic cell xenotransplantation in nonhuman primate is complicated by a thrombotic microangiopathy. Bone Marrow Transplant (in press)

35. Hawley R J, Abraham S, Akiyoshi D E, et al. Xenogeneic bone marrow

35 transplantation: I. Cloning, expression, and species specificity of porcine IL-3 and granulocyte-macrophage colony-stimulating factor. Xenotransplantation 1997; 4: 103.

36. Giovino M A, Hawley R J, Dickerson W M, et al. Xenogeneic bone marrow transplantation: II. Porcine-specific growth factors enhance porcine bone marrow engraftment in an in vitro primate microenvironment. Xenotransplantation 1997; 4: 112.

37. Buhler L, Awwad M, Treter S, et al. Pig hematopoietic cell chimerism in baboons conditioned with a nonmyeloablative regimen and CD154 blockade. Transplantation. In press.

38. Dalmasso A P, He T, Benson B A. Human IgM xenoreactive natural antibodies can induce resistance of porcine endothelial cells to complement-mediated injury. Xenotransplantation 1996; 3: 54.

39. Koren E, Neethling F A, Ye Y, et al. Heterogeneity of preformed human antipig xenogeneic antibodies. Transplant Proc 1992; 24: 598.

40. Koren E, Milotic F, Neethling F A, et al. Monoclonal antiidiotypic antibodies neutralize cytotoxic effects of anti-alphaGal antibodies. Transplantation 1996; 62: 837.

41. Neethling F A, Joziasse D, Bovin N, Cooper D K C, Oriol R. The reducing end of alpha Gal oligosaccharides contributes to their efficiency in blocking natural antibodies of human and baboon sera. Transpl Int 1996; 9: 98.

42. Goldberg L C, Lee J, Cairns T, et al. Polymorphism within the human anti-pig repertoire. Transplant Proc 1996; 28: 549.

43. Parker W, Lateef J, Everett M L, Plaft J L. Specificity of xenoreactive anti-Gal alpha 1-3Gal IgM for alpha-galactosyl ligands. Glycobiology 1996; 6: 499.

44. McMorrow I M, Comrack C A, Sachs D H, DerSimonian H. Heterogeneity of human anti-pig natural antibodies cross- reactive with the Gal(alpha1,3)Galactose epitope. Transplantation 1997; 64: 501.

45. McKane W, Lee J, Preston R, et al. Polymorphism in the human anti-pig natural antibody repertoire: implications for antigen-specific immunoadsorption. Transplantation 1998; 66: 626.

46. Basker M, Alwayn I P J, Buhler L, et al. Clearance of mobilized porcine peripheral blood progenitor cells is delayed by depletion of the phagocytic reticuloendothelial system in baboons. Transplantation (in press)

what is claimed is:

1. A process for reducing a natural antibody response in a primate recipient against a graft from a non-primate donor, comprising administering to said recipient an effective amount of one or more galactosyl-α-1,3-galactose moieties linked to a carrier.

2. The process of claim 1 wherein said primate is a human being.

3. The process of claim 1 wherein said carrier is a protein.

4. The process of claim 3 wherein said protein is selected from the group consisting of bovine serum albumin and human serum albumin.

5. The process of claim 1 wherein said carrier is a polymer.

6. The process of claim 1 wherein said carrier is linked to a plurality of galactosyl-α-1 ,3-galactose moieties.

7. The process of claim 6 wherein said plurality of galactosyl-α-1,3-galactose moieties comprises at least two chemically different galactosyl-α-1,3-galactose moieties.

8. The process of claim 1 further comprising administration to said recipient of an effective amount of an immunosuppressive agent.

9. The process of claim 8 wherein said immunosuppressive agent comprises an inhibitor of a co-stimulatory pathway.

10. The process of claim 9 wherein said co-stimulatory inhibitor is an inhibitor of CD40-CD154 interaction.

11. The process of claim 10 wherein said inhibitor is a member selected from the group consisting of a soluble ligand of CD154, a soluble ligand of CD40, a receptor for CD154 and a receptor for CD40.

12. The process of claim 9 wherein said co-stimulatory inhibitor is an inhibitor of a CD28-B7 interaction.

13. The process of claim 10 wherein said inhibitor is a member selected from the group consisting of a soluble ligand of CD28, a soluble ligand of B7, a receptor for CD28 and a receptor for B7.

14. The process of claim 10 wherein said inhibitor is selected from the group consisting of CTLA4 fusion protein and CTLA4Ig.

15. The process of claim 8 wherein said immunosuppressive agent comprises a T cell depleting agent.

16. The process of claim 15 wherein said T cell depleting agent is a lymphocytic agent.

17. The process of claim 15 wherein said T cell depleting agent is an agent that induces apoptosis in T cells.

18. The process of claim 8 wherein said immunosuppressive agent comprises both an inhibitor of a co-stimulatory pathway and a T cell depleting agent.

19. The process of claim 8 wherein said immunosuppressive agent comprises an antibody.

20. The process of claim 19 wherein said antibody is an anti-CD154 antibody.

21. The process of claim 20 wherein said anti-CD154 antibody is 5c8.

22. The process of claim 19 wherein said antibody is an anti-CD2 antibody.

23. The process of claim 19 wherein said antibody is MEDI-507.

24. The process of claim 1 further comprising administering to said recipient an effective amount of hematopoietic stem cells.

25. The process of claim 24 wherein said hematopoietic stem cells are allogeneic to the donor.

26. The process of claim 25 wherein said hematopoietic stem cells are derived from the donor.

27. The process of claim 1 further comprising administering to said recipient an effective amount of an immunosuppressive agent and an effective amount of hematopoietic stem cells.

28. The process of claim 27 wherein said hematopoietic stem cells are allogeneic to the donor.

29. The process of claim 27 wherein said hematopoietic stem cells are derived from the donor.

30. The process of claim 9 further comprising administering to the recipient a sample of donor thymic tissue.

31. The process of claim 30 wherein said thymic tissue is fetal thymic tissue.

32. The process of claim 30 wherein said thymic tissue is porcine thymic tissue.

33. The process of claim 30 wherein said thymic tissue is porcine fetal thymic tissue.

34. The process of claim 30 wherein said thymic tissue is transplanted into said recipient prior to or simultaneously with the graft against which rejection is to be reduced.

35. The process of claim 9 further comprising introducing into the recipient a sample of a composite thymo-organ.

36. The process of claim 35 wherein said composite thymo-organ is selected from the group consisting of thymo-kidney and thymo-heart.

37. The process of claim 35 wherein said composite thymo-organ is a porcine thymo-organ.

38. The process of claim 9 further comprising introducing into the recipient a sample of an islet-thymo-organ.

39. The process of claim 38 wherein the thymo-organ of said islet-thymo-organ is selected from the group consisting of thymo-kidney and thymo-heart.

40. The process of claim 38 wherein said islet-thymo-organ is a porcine islet-thymo-organ.
